SEQUENCE LISTING (125 sequences)

SEQ ID NO: 1 - protein, 10 aa, Artificial sequence
  Synthetic peptide
SFNSYELGSL

SEQ ID NO: 2 - protein, 10 aa, Artificial sequence
  Synthetic peptide
TFNSYELGSL

SEQ ID NO: 3 - protein, 11 aa, Artificial sequence
  Synthetic peptide
AFNSNYELGSL

SEQ ID NO: 4 - protein, 10 aa, Artificial sequence
  Synthetic peptide
SFNSYELGTL

SEQ ID NO: 5 - protein, 10 aa, Artificial sequence
  Synthetic peptide
STNSYELGSL

SEQ ID NO: 6 - protein, 10 aa, Artificial sequence
  Synthetic peptide
SFNSFELGSL

SEQ ID NO: 7 - protein, 9 aa, Artificial sequence
  Synthetic peptide
SNSYDLGSL

SEQ ID NO: 8 - protein, 10 aa, Artificial sequence
  Synthetic peptide
SFNSYELPSL

SEQ ID NO: 9 - protein, 10 aa, Artificial sequence
  Synthetic peptide
TFNSYELGTL

SEQ ID NO: 10 - protein, 10 aa, Artificial sequence
  Synthetic peptide
SFNSYEIGSV

SEQ ID NO: 11 - protein, 10 aa, Artificial sequence
  Synthetic peptide
SFNSYEVGSI

SEQ ID NO: 12 - protein, 10 aa, Artificial sequence
  Synthetic peptide
SFNSYELGSV

SEQ ID NO: 13 - protein, 10 aa, Artificial sequence
  Synthetic peptide
SFNSYELGSI

SEQ ID NO: 14 - protein, 10 aa, Artificial sequence
  Synthetic peptide
SFNSYEIGSL

SEQ ID NO: 15 - protein, 10 aa, Artificial sequence
  Synthetic peptide
SFNSYEVGSL

SEQ ID NO: 16 - protein, 10 aa, Artificial sequence
  Synthetic peptide
AFNSYELGSL

SEQ ID NO: 17 - protein, 6 aa, Artificial sequence
  Synthetic peptide
YDLGSL

SEQ ID NO: 18 - protein, 6 aa, Artificial sequence
  Synthetic peptide
FDLGSL

SEQ ID NO: 19 - protein, 6 aa, Artificial sequence
  Synthetic peptide
YDIGSL

SEQ ID NO: 20 - protein, 6 aa, Artificial sequence
  Synthetic peptide
YDVGSL

SEQ ID NO: 21 - protein, 6 aa, Artificial sequence
  Synthetic peptide
YDLPSL

SEQ ID NO: 22 - protein, 6 aa, Artificial sequence
  Synthetic peptide
YDLGLL

SEQ ID NO: 23 - protein, 6 aa, Artificial sequence
  Synthetic peptide
YDLGSI

SEQ ID NO: 24 - protein, 6 aa, Artificial sequence
  Synthetic peptide
YDLGSV

SEQ ID NO: 25 - protein, 4 aa, Artificial sequence
  Synthetic peptide
IGSL

SEQ ID NO: 26 - protein, 4 aa, Artificial sequence
  Synthetic peptide
VGSL

SEQ ID NO: 27 - protein, 4 aa, Artificial sequence
  Synthetic peptide
LPSL

SEQ ID NO: 28 - protein, 4 aa, Artificial sequence
  Synthetic peptide
LGLL

SEQ ID NO: 29 - protein, 4 aa, Artificial sequence
  Synthetic peptide
LGSI

SEQ ID NO: 30 - protein, 4 aa, Artificial sequence
  Synthetic peptide
LGSV

SEQ ID NO: 31 - protein, 10 aa, Artificial sequence
  Synthetic peptide
ELGSLQAEDD

SEQ ID NO: 32 - protein, 10 aa, Artificial sequence
  Synthetic peptide
ELGSLQAEDE

SEQ ID NO: 33 - protein, 9 aa, Artificial sequence
  Synthetic peptide
SFNSYELGS

SEQ ID NO: 34 - protein, 10 aa, Artificial sequence
  Synthetic peptide
SFNSYELGSL

SEQ ID NO: 35 - protein, 10 aa, Artificial sequence
  Synthetic peptide
ELGSLQAEDD

SEQ ID NO: 36 - protein, 13 aa, Artificial sequence
  Synthetic peptide
GSFNSYELGSLGG

SEQ ID NO: 37 - protein, 10 aa, Artificial sequence
  Synthetic peptide
SANSYELGSL

SEQ ID NO: 38 - protein, 8 aa, Artificial sequence
  Synthetic peptide
NSYELGSL

SEQ ID NO: 39 - protein, 11 aa, Artificial sequence
  Synthetic peptide
ALSTERGKTLV

SEQ ID NO: 40 - protein, 11 aa, Artificial sequence
  Synthetic peptide
ALSTDRGKTLV

SEQ ID NO: 41 - protein, 11 aa, Artificial sequence
  Synthetic peptide
ALTSDRGKTLV

SEQ ID NO: 42 - protein, 11 aa, Artificial sequence
  Synthetic peptide
ALTTDRGKSLV

SEQ ID NO: 43 - protein, 11 aa, Artificial sequence
  Synthetic peptide
ALTTDRPKTLV

SEQ ID NO: 44 - protein, 11 aa, Artificial sequence
  Synthetic peptide
ALTTDRGRTLV

SEQ ID NO: 45 - protein, 11 aa, Artificial sequence
  Synthetic peptide
ALTTDKGKTLV

SEQ ID NO: 46 - protein, 11 aa, Artificial sequence
  Synthetic peptide
ALTTDKGKTLV

SEQ ID NO: 47 - protein, 10 aa, Artificial sequence
  Synthetic peptide
VLMRAAEEPV

SEQ ID NO: 48 - protein, 10 aa, Artificial sequence
  Synthetic peptide
QSMRSEDEAK

SEQ ID NO: 49 - protein, 9 aa, Artificial sequence
  Synthetic peptide
AFNSYELGS

SEQ ID NO: 50 - protein, 9 aa, Artificial sequence
  Synthetic peptide
QGFEKKTGV

SEQ ID NO: 51 - protein, 9 aa, Artificial sequence
  Synthetic peptide
DNNGTYGKI

SEQ ID NO: 52 - protein, 6 aa, Artificial sequence
  Synthetic peptide
KNLIDS

SEQ ID NO: 53 - protein, 8 aa, Artificial sequence
  Synthetic peptide
VKSPRDYS

SEQ ID NO: 54 - protein, 9 aa, Artificial sequence
  Synthetic peptide
VKSPCRDYS

SEQ ID NO: 55 - protein, 8 aa, Artificial sequence
  Synthetic peptide
IKSPRLYS

SEQ ID NO: 56 - protein, 6 aa, Artificial sequence
  Synthetic peptide
KNLIDS

SEQ ID NO: 57 - protein, 11 aa, Artificial sequence
  Synthetic peptide
PKVKSPRDYSN

SEQ ID NO: 58 - protein, 10 aa, Artificial sequence
  Synthetic peptide
SFNSYELGSL

SEQ ID NO: 59 - protein, 10 aa, Artificial sequence
  Synthetic peptide
TFNSYELGSL

SEQ ID NO: 60 - protein, 11 aa, Artificial sequence
  Synthetic peptide
AFNSNYELGSL

SEQ ID NO: 61 - protein, 10 aa, Artificial sequence
  Synthetic peptide
SFNSYELGTL

SEQ ID NO: 62 - protein, 10 aa, Artificial sequence
  Synthetic peptide
STNSYELGSL

SEQ ID NO: 63 - protein, 10 aa, Artificial sequence
  Synthetic peptide
SFNSFELGSL

SEQ ID NO: 64 - protein, 9 aa, Artificial sequence
  Synthetic peptide
SNSYDLGSL

SEQ ID NO: 65 - protein, 10 aa, Artificial sequence
  Synthetic peptide
SFNSYELPSL

SEQ ID NO: 66 - protein, 10 aa, Artificial sequence
  Synthetic peptide
TFNSYELGTL

SEQ ID NO: 67 - protein, 10 aa, Artificial sequence
  Synthetic peptide
SFNSYEIGSV

SEQ ID NO: 68 - protein, 10 aa, Artificial sequence
  Synthetic peptide
SFNSYEVGSI

SEQ ID NO: 69 - protein, 10 aa, Artificial sequence
  Synthetic peptide
SFNSYELGSV

SEQ ID NO: 70 - protein, 10 aa, Artificial sequence
  Synthetic peptide
SFNSYELGSI

SEQ ID NO: 71 - protein, 10 aa, Artificial sequence
  Synthetic peptide
SFNSYEIGSL

SEQ ID NO: 72 - protein, 10 aa, Artificial sequence
  Synthetic peptide
SFNSYEVGSL

SEQ ID NO: 73 - protein, 10 aa, Artificial sequence
  Synthetic peptide
AFNSYELGSL

SEQ ID NO: 74 - protein, 6 aa, Artificial sequence
  Synthetic peptide
YDLGSL

SEQ ID NO: 75 - protein, 6 aa, Artificial sequence
  Synthetic peptide
FDLGSL

SEQ ID NO: 76 - protein, 6 aa, Artificial sequence
  Synthetic peptide
YDIGSL

SEQ ID NO: 77 - protein, 6 aa, Artificial sequence
  Synthetic peptide
YDVGSL

SEQ ID NO: 78 - protein, 6 aa, Artificial sequence
  Synthetic peptide
YDLPSL

SEQ ID NO: 79 - protein, 6 aa, Artificial sequence
  Synthetic peptide
YDLGLL

SEQ ID NO: 80 - protein, 6 aa, Artificial sequence
  Synthetic peptide
YDLGSI

SEQ ID NO: 81 - protein, 6 aa, Artificial sequence
  Synthetic peptide
YDLGSV

SEQ ID NO: 82 - protein, 4 aa, Artificial sequence
  Synthetic peptide
IGSL

SEQ ID NO: 83 - protein, 4 aa, Artificial sequence
  Synthetic peptide
VGSL

SEQ ID NO: 84 - protein, 4 aa, Artificial sequence
  Synthetic peptide
LPSL

SEQ ID NO: 85 - protein, 4 aa, Artificial sequence
  Synthetic peptide
LGLL

SEQ ID NO: 86 - protein, 4 aa, Artificial sequence
  Synthetic peptide
LGSI

SEQ ID NO: 87 - protein, 4 aa, Artificial sequence
  Synthetic peptide
LGSV

SEQ ID NO: 88 - protein, 11 aa, Artificial sequence
  Synthetic peptide
ALSTERGKTLV

SEQ ID NO: 89 - protein, 11 aa, Artificial sequence
  Synthetic peptide
ALSTDRGKTLV

SEQ ID NO: 90 - protein, 11 aa, Artificial sequence
  Synthetic peptide
ALTSDRGKTLV

SEQ ID NO: 91 - protein, 11 aa, Artificial sequence
  Synthetic peptide
ALTTDRGKSLV

SEQ ID NO: 92 - protein, 11 aa, Artificial sequence
  Synthetic peptide
ALTTDRPKTLV

SEQ ID NO: 93 - protein, 11 aa, Artificial sequence
  Synthetic peptide
ALTTDRGRTLV

SEQ ID NO: 94 - protein, 11 aa, Artificial sequence
  Synthetic peptide
ALTTDKGKTLV

SEQ ID NO: 95 - protein, 11 aa, Artificial sequence
  Synthetic peptide
ALTTDKGKTLV

SEQ ID NO: 96 - protein, 10 aa, Artificial sequence
  Synthetic peptide
VLMRAAEEPV

SEQ ID NO: 97 - protein, 10 aa, Artificial sequence
  Synthetic peptide
QSMRSEDEAK

SEQ ID NO: 98 - protein, 9 aa, Artificial sequence
  Synthetic peptide
AFNSYELGS

SEQ ID NO: 99 - protein, 9 aa, Artificial sequence
  Synthetic peptide
QGFEKKTGV

SEQ ID NO: 100 - protein, 9 aa, Artificial sequence
  Synthetic peptide
DNNGTYGKI

SEQ ID NO: 101 - protein, 6 aa, Artificial sequence
  Synthetic peptide
KNLIDS

SEQ ID NO: 102 - protein, 8 aa, Artificial sequence
  Synthetic peptide
VKSPRDYS

SEQ ID NO: 103 - protein, 9 aa, Artificial sequence
  Synthetic peptide
VKSPCRDYS

SEQ ID NO: 104 - protein, 8 aa, Artificial sequence
  Synthetic peptide
IKSPRLYS

SEQ ID NO: 105 - protein, 6 aa, Artificial sequence
  Synthetic peptide
KNLIDS

SEQ ID NO: 106 - protein, 11 aa, Artificial sequence
  Synthetic peptide
PKVKSPRDYSN

SEQ ID NO: 107 - protein, 11 aa, Artificial sequence
  Synthetic peptide
CSFNSYELGSL

SEQ ID NO: 108 - protein, 12 aa, Artificial sequence
  Synthetic peptide
CYGRKKRRQRRR

SEQ ID NO: 109 - protein, 11 aa, Artificial sequence
  Synthetic peptide
CSFNSYELGSL

SEQ ID NO: 110 - protein, 11 aa, Artificial sequence
  Synthetic peptide
CSFNSYELGSL

SEQ ID NO: 111 - protein, 12 aa, Artificial sequence
  Synthetic peptide
CYGRKKRRQRRR

SEQ ID NO: 112 - protein, 11 aa, Artificial sequence
  Synthetic peptide
CELGSLQAEDD

SEQ ID NO: 113 - protein, 10 aa, Artificial sequence
  Synthetic peptide
CSFNSYELGS

SEQ ID NO: 114 - protein, 11 aa, Artificial sequence
  Synthetic peptide
CSFNSYELGSL

SEQ ID NO: 115 - protein, 12 aa, Artificial sequence
  Synthetic peptide
CYGRKKRRQRRR

SEQ ID NO: 116 - protein, 11 aa, Artificial sequence
  Synthetic peptide
CELGSLQAEDD

SEQ ID NO: 117 - protein, 24 aa, Artificial sequence
  Synthetic peptide
GSFNSYELGSLGGYGRKKRRQRRR

SEQ ID NO: 118 - protein, 11 aa, Artificial sequence
  Synthetic peptide
CSANSYELGSL

SEQ ID NO: 119 - protein, 9 aa, Artificial sequence
  Synthetic peptide
CNSYELGSL

SEQ ID NO: 120 - protein, 55 aa, Artificial sequence
  Synthetic peptide
PKVKSPRDYSNFDQEFLNEKARLSYSDKNLIDSMDQSAFAGFSFVNPKFEHLLED

SEQ ID NO: 121 - protein, 8 aa, Artificial sequence
  Synthetic peptide
VKSPRDYS

SEQ ID NO: 122 - protein, 11 aa, Artificial sequence
  Synthetic peptide
PKVKSPRDYSN

SEQ ID NO: 123 - protein, 9 aa, Artificial sequence
  Synthetic peptide
VKSPCRDYS

SEQ ID NO: 124 - protein, 6 aa, Artificial sequence
  Synthetic peptide
IKSPXS

SEQ ID NO: 125 - protein, 11 aa, Artificial sequence
  Synthetic peptide
YGRKKRRQRRR

(19) United States
(12) Patent Application Publication
(10) Pub. No.: US 2013/0012447 A1
(43) Pub. Date: Jan. 10, 2013

(54) PROTEIN KINASE C-DELTA INHIBITORS THAT PROTECT AGAINST CELLULAR INJURY AND INFLAMMATION AND PROMOTE ASTROCYTE PROLIFERATION

(76) Inventor: Eiketsu Sho, Hayward, CA (US)

(21) Appl. No.: 12/675,761

(22) PCT Filed: Aug. 27, 2008
(86) PCT No.: PCT/US08/74509

§ 371 Date: May 18, 2012

Related U.S. Application Data
(60) Provisional application No. 60/968,283, filed on Aug. 27, 2007.

(51) Int. Cl. A61K 38/16; A61P 25/00; A61K 38/07; A61K 38/10; A61K 38/08

(52) U.S. Cl. 514/17.7

(57) ABSTRACT

The invention relates to the use of δPKC inhibitor peptides to treat brain injury, particularly traumatic brain injury (TBI). In one embodiment, peptide that specifically inhibit δPKC are used to protect neurological tissue by promoting astrocyte proliferation.

### CROSS-REFERENCE TO RELATED APPLICATIONS

This application claims priority from U.S. provisional application 60/968,283 filed Oct. 27, 2007. The entire contents of this document are incorporated herein by reference.

### TECHNICAL FIELD

The invention relates to the use of δPKC inhibitor peptides to treat brain injury, particularly traumatic brain injury (TBI). In one embodiment, δPKC inhibitor peptides are used to protect neurological tissue by promoting astrocyte proliferation.

### BACKGROUND ART

Astrocytes play both a protective role and yet have been associated with a number of disease states. For example, astrocytes are one of the cell types from which gliomas are thought to originate. Astrocytes are essential cells in neuronal system to contribute a special unit (capillary-astrocyte-neuron unit) in brain so as to support neuronal system function. Astrocytes respond to traumatic brain injury (TBI) by altered gene expression, hypertrophy and proliferation that occur in a gradated fashion in relation to the severity of the injury. Both beneficial and detrimental effects have been attributed to reactive astrocytes. Studies indicate that the reactive astrocytes play essential roles in preserving neural tissue and restricting inflammation after moderate focal brain injury. (See, e.g., Myer et al., Brain (2006) 129:2761-2772, and Kernie, et al., J. Neurosci-Res. (2001) 66(3): 317-26.)

The persistence of neural stem cells into adulthood has been an area of intense investigation in recent years. Studies demonstrate that there is significant proliferation of neural precursors in response to traumatic brain injury in areas both proximal and distal to the injury site. The fate of the proximal proliferation is almost exclusively astrocytic at 60-days post injury and demonstrates that newly generated cells make up much of the astrogliotic scar. These data demonstrate that neural proliferation plays key roles in the remodeling that occurs after traumatic brain injury and suggests a mechanism as to how functional recovery after traumatic brain injuries continues to occur long after the injury itself.

The contribution of brain edema to brain swelling in cases of traumatic brain injury (TBI) remains a critical problem. Inflammatory reactions may play a fundamental role in brain swelling following a head injury. The studies suggest that the acute response to severe head trauma with early edema formation is likely to be associated with inflammatory events which might be triggered by activated microglia and infiltrating lymphocytes. It is difficult to overestimate the clinical significance of these observations, as the early and targeted treatment of patients with severe head injuries with immunosuppressive medication may result in a far more favorable outcome.

Protein kinase C (“PKC”) is a key enzyme in signal transduction involved in a variety of cellular functions, including cell growth, regulation of gene expression, and ion channel activity. The PKC family of isozymes includes at least 11 different protein kinases that can be divided into at least three subfamilies based on their homology and sensitivity to activators. Each isozyme includes a number of homologous (“conserved” or “C”) domains interspersed with isozyme-unique (“variable” or “V”) domains. Members of the “classical” or “cPKC” subfamily, α, β_{I}, β_{II} and γPKC, contain four homologous domains (C1, C2, C3 and C4) and require calcium, phosphatidylserine, and diacylglycerol or phorbol esters for activation. Members of the “novel” or “nPKC” subfamily, δ, ε, η and .θPKC, lack the C2 homologous domain and do not require calcium for activation. Finally, members of the “atypical” or “αPKC” subfamily, ζ and λ/iPKC, lack both the C2 and one-half of the C1 homologous domains and are insensitive to diacylglycerol, phorbol esters and calcium.

The role the PKCs play in astrocyte recruitment and proliferation is poorly understood. One group reported that δPKC played a role in astrocyte migration. Renault-Mihara et al. Mole. Bio. Cell. (2006) 17:5141-5152. This study suffers from a number of defects, however, and its conclusions are not well supported, primarily because the δPKC inhibitors (e.g., rottlerin) used were not specific for the delta isozyme. Soltoff reported that rottlerin was an inappropriate and ineffective inhibitor of δPKC. Trends Pharmacol Sci. (2007) 28(9):453-8. Moreover, Mihara et al. note that astrocytes are one of the cell types from which gliomas, the main primitive brain tumors of adulthood, originate.

### SUMMARY OF THE INVENTION

The disclosed invention relates to the use of compounds that specifically inhibit δPKC to treat traumatic brain injury (TBI). Preferably the compounds are peptides that specifically inhibit δPKC.

One embodiment of the disclosed invention relates to a method to treat traumatic brain injury, comprising identifying a subject suffering from a traumatic brain injury (TBI) by identifying the presence of TBI symptoms, and administering a therapeutically effective amount of a peptide that specifically inhibits δPKC activity, whereby astrocyte activity is increased and one or more of the symptoms of TBI are reduced. The administering can occur within 1 to 5 hours of the TBI and it can encompass a peptide comprising 4 to 25 residues of the first variable region of δPKC. Alternatively, the peptide can comprise 4 to 25 residues of the fifth variable region of δPKC. Another aspect of this embodiment includes administering the δPKC peptide antagonist linked to a moiety effective to facilitate transport across a cell membrane. Examples of suitable moieties can be elected from the group consisting of a Tat-derived peptide, an Antennapedia carrier peptide, and a polyarginine peptide. In a preferred embodiment, the peptide is KAI-9803. In another aspect of the invention, the symptoms comprise increase of glucose utilization, energy-dependent membrane depolarization, or cerebral metabolic rate changes.

Another embodiment of the disclosed invention relates to a method of stimulating astrocyte activity, comprising providing a therapeutically effective amount of a δPKC inhibitory peptide to a population of astrocytes, whereby astrocyte proliferation is increased relative to a population of astrocytes not provided the δPKC inhibitory peptide. In one aspect, the peptide comprises 4 to 25 residues of the first variable region of δPKC. In a preferred embodiment, the peptide administered is KAI-9803. In another aspect, the peptide comprises 4 to 25 residues of the fifth variable region of δPKC. In another aspect, the administering step comprises administering the δPKC peptide antagonist linked to a moiety effective to facilitate transport across a cell membrane. In still another aspect of the invention, the moiety is selected from the group consisting of a Tat-derived peptide, an Antennapedia carrier peptide, and a polyarginine peptide.

### BRIEF DESCRIPTION OF THE DRAWINGS

FIG. 1 shows a schematic representation of surgical procedures to produce MCAO model. RCCA: right carotid artery; LECA: left external carotid artery; LICA: left internal carotid artery.

FIG. 2 shows a schematic representation of penumbra area for cell calculation. Grey color indicates ischemia core area. White color with red line indicates penumbra. Number of 1-5 indicates the area of images for cell counting and calculation.

FIG. 3 shows a bar graph illustrating the effect of KAI 9803 on rat tMCAO. *p<0.01 vs. saline at each time point.

FIG. 4 shows a line graph illustrating the effect of KAI-9803 protection against neuron damage in penumbra. S-N-C: contralateral neuron/astrocyte density (cortex) in saline group; S-N-i: ipsilateral neuron density in penumbra in saline group; K-N-i: ipsilateral neuron density in penumbra in KAI-9803 group.

FIG. 5 shows a line graph illustrating the effect of KAI-9803 protection against astrocyte damage in penumbra. S-N-C: contralateral neuron/astrocyte density (cortex) in saline group; S-N-i: ipsilateral astrocyte density in penumbra in saline group; K-N-i: ipsilateral astrocyte density in penumbra in KAI-9803 group.

FIG. 6 shows a line graph illustrating the effect of KAI-9803 on protection against capillary damage in ischemia core.

FIG. 7 shows a line graph illustrating the effect of KAI-9803 on protection against capillary damage in penumbra.

FIG. 8 shows a line graph illustrating the effect of KAI-9803 on protection against macrophage infiltration in penumbra

FIG. 9 shows a line graph illustrating the effect of treatment of KAI-9803 enhancement astrocyte proliferation in penumbra.

### DETAILED DESCRIPTION OF THE INVENTION

The disclosed invention relates to the use of compounds that specifically inhibit δPKC to treat traumatic brain injury (TBI). In a preferred embodiment, the inhibitory compounds are peptides that specifically inhibit δPKC. A “δPKC inhibitor” is any compound, including small molecules and peptides, which is capable of inhibiting the enzymatic activity and other functional activities of δPKC isozyme. A specific δPKC inhibitor is any compound which measurably inhibits δPKC isozyme over another. A particular aspect of the invention relates to the modulatory impact of δPKC inhibitory compounds on astrocyte activity. The term “astrocyte activity” encompasses components of astrocyte metabolism, astrocyte migration, (e.g., infiltration of a site), as well as astrocyte proliferation. In a preferred embodiment, the TBI treated by the described method is not caused by stroke. Unless otherwise specified, the TBI discussed below is not caused by stroke. The disclosed invention further contemplates diagnostic measures of brain injury and treatments of TBI through the monitoring of astrocyte activity.

While not wishing to be bound by any particular theory, a preferred embodiment of the presently disclosed invention relates to increasing astrocyte activity in the brain of subjects suffering from TBI. Astrocytes are glial cells in the brain. Astrocytes play a number of different roles in the brain, For example, astrocytes comprise a portion of the physical structure of the brain, by forming part of the blood-brain barrier. Astrocytes nourish nervous tissue, for example by providing neurons with nutrients. Astrocytes have also been reported to play a role in neurotransmitter reuptake and release. Astrocytes are thought to regulate ion concentrations within the interstitial space and regulate blood flow. Perhaps most importantly, astrocytes are thought to play a role in repairing damage to the brain. For example, following brain infarction or trauma and the development of necrotic tissue at the site of injury, astrocytes and other cells are thought to colonize the damages spaces and stabilize the damaged region and promote its repair. The use of δPKC inhibitory peptides is thought to stimulate the repair role astrocytes and other cells play in response to TBI. In one embodiment, the δPKC inhibitory peptides are used to induce astrocyte proliferation or infiltration of a traumatic brain injury in subjects in need thereof.

### Mechanisms of Injury

The following mechanisms of injury represent the most common cause of TBI. These mechanisms include: open head injury, closed head injury, deceleration injuries, chemical/toxic, hypoxia, tumors, infections and stroke. The following mechanisms are provided for illustrative purposes and the description of these mechanisms is not intended to limit the scope of the claims.

Open head trauma resulting from wounds such as bullet wounds and other penetrations of the skull represent a major cause of TBI. Closed head injuries resulting from falls, motor vehicle crashes, and concussions caused by explosions, blunt force trauma, or other external forces are another major cause of TBI. Deceleration injuries (diffuse axonal injury) occur when a skull moving in space decelerates while the brain encased therein continues to move at speed. Differential movement of the skull and brain can result in shearing, contusion, and brain swelling. This shearing can damage axons and lead to neuronal death. Exposure of the brain to various chemicals can cause TBI, as well as hypoxia, tumors, infections, and stroke.

Symptoms of TBI include a significant increase of glucose utilization within the first 30 minutes post-injury, after which glucose uptake diminishes and then remains low for about 5-10 days. TBI has also been reported to increase membrane permeability and consecutive edema formation. ATP-stores are depleted and there is a failure of energy-dependent membrane depolarization. In addition to glycolytic disturbances TBI can also lead to impairment of oxidative metabolism following brain trauma. For example, severely head injured patients frequently show cerebral lactic acidosis. Cerebral hemodynamics change significantly post injury, and the pattern of these changes depends upon the type of injury and its severity. TBI has also been reported to cause a rapid release of glutamate the predominant excitatory neurotransmitter in the central nervous system. (See Madilians & Giza, Indian Journal of Neurotrama (2006) 3:9-17. Cerebral hemodynamics and metabolism can be measured using positron emission tomography (PET), as discussed in Yamaki et al. J. Nucl. Med. (1996) 37(7):1170-2. Metabolic measurements include regional cerebral blood flow (rCBF), oxygen extraction fraction (rOEF), cerebral blood volume (rCBV), cerebral metabolic rate for oxygen (rCMRO2), cerebral metabolic rate for glucose (rCMRglc) and cerebral metabolic ratio (rCMRO2/rCMRglc). Use of the Glasgow Coma Scale scores and computed tomography (CT) can also be used to diagnose TBI.

### δPKC Inhibitors

The invention includes compounds, such as small molecules and peptides that inhibit δPKC activity. Small molecule inhibitors of PKC are described in U.S. Pat. Nos. 5,141,957, 5,204,370, 5,216,014, 5,270,310, 5,292,737, 5,344,841, 5,360,818, 5,432,198, 5,380,746, and 5,489,608, (European Patent 0,434,057), all of which are hereby incorporated by reference in their entirety. These molecules belong to the following classes: N,N′-Bis-(sulfonamido)-2-amino-4-iminonaphthalen-1-ones; N,N′-Bis-(amido)-2-amino-4-iminonaphthalen-1-ones; vicinal-substituted carbocyclics; 1,3-dioxane derivatives; 1,4-Bis-(amino-hydroxyalkylamino)-anthraquinones; furo-coumarinsulfonamides; Bis-(hydroxyalkylamino)-anthraquinones; and N-aminoalkyl amides, 2-[1-(3-Aminopropyl)-1H-indol-3-yl]-3-(1H-indol-3-yl)maleimide, 2-[1-[2-(1-Methylpyrrolidino)ethyl]-1H-indol-3-yl]-3-(1H-indol-3-yl)maleimide, Go 7874. Other known small molecule inhibitors of PKC are described in the following publications (Fabre, S., et al. 1993. Bioorg. Med. Chem. 1, 193, Toullec, D., et al. 1991. J. Biol. Chem. 266, 15771, Gschwendt, M., et al. 1996. FEBS Lett. 392, 77, Merritt, J. E., et al. 1997. Cell Signal 9, 53., Birchall, A. M., et al. 1994. J. Pharmacol. Exp. Ther. 268, 922. Wilkinson, S. E., et al. 1993. Biochem. J. 294, 335., Davis, P. D., et al. 1992. J. Med. Chem. 35, 994), and belong to the following classes: 2,3-bis(1H-Indol-3-yl)maleimide (Bisindolylmaleimide IV); 2-[1-(3-Dimethylaminopropyl)-5-methoxyindol-3-yl]-3-(1H-indol-3-yl) maleimide (Go 6983); 2-{8-[(Dimethylamino)methyl]-6,7,8,9-tetrahydropyrido [1,2-a]indol-3-yl}-3-(1-methyl-1H-indol-3-yl)maleimide (Ro-32-0432); 2-[8-(Aminomethyl)-6,7,8,9-tetrahydropyrido[1,2-a]indol-3-yl]-3-(1-methyl-1H-indol-3-yl)maleimide (Ro-31-8425); and 3-[1-[3-(Amidinothio)propyl-1H-indol-3-yl]-3-(1-methyl-1H-indol-3-yl)maleimide Bisindolylmaleimide IX, Methanesulfonate (Ro-31-8220) all of which are also hereby incorporated by reference in their entirety.

The invention also contemplates the use of peptides effective to inhibit δPKC and which are capable of stimulation astrocyte proliferation or TBI site infiltration. A discussed herein, the inhibitory peptide is frequently referred to as the “cargo” peptide. A variety of inhibitory δPKC peptides have been described in the art, such as those described in U.S. Pat. No. 6,855,693, U.S. Patent Application No. 20050215483 and U.S. Provisional Patent Application Nos. 60/881,419 and 60/945,285, all of which are hereby incorporated by reference.

The δPKC inhibitory peptides act as translocation inhibitors of δPKC, which serve to reduce δPKC activity in treated cells. It will be appreciated that the inhibitory peptides can be used in native form or modified by conjugation to a carrier to facilitate cellular uptake of the PKC inhibitory peptides. Examples of modifications to the peptides can be found in U.S. Provisional Application Nos. 60/881,419 and 60/945,285, both of which are hereby incorporated by reference in their entirety. For example, when a carrier and cargo peptide are linked via a disulfide bond, a homoCys residue can be used in place of Cys. Additionally, capping the amino and carboxy termini of the cargo, the carrier, or both peptides are contemplated, as fully explained in the referenced applications.

Peptides derived from the first and fifth variable regions of the enzyme are contemplated for use as inhibitory peptides with the methods described herein. The peptides are preferably 4 to 25 residues in length, more preferably 6 to 25 residues in length, and still more preferably 6 to 12 residues in length. Another preferred embodiment contemplates the use of peptides from 6 to 8, 9 10, 11, 12, 13, 14, 15, 16, 17, 18, 19, 20, 21, 22, 23, 24, or 25 residues in length, excluding residues used to join the cargo and carrier sequences, such as a Gly-Gly dimer, or one or more Cys residue for forming disulfide bonds.

It will be appreciated that peptides homologous to the native sequences and peptides having conservative amino acid substitutions and/or juxtapositions, as well as fragments that retain activity, are within the scope of peptides contemplated. For example, one or more amino acids (preferably no more than two) can be substituted, changing between R and K; between V, L, I, R and D; and/or between G, A, P and N. Thus, the term “δPKC inhibit peptide” contemplates the native sequence and all modifications, derivations, fragments, combinations, and hybrids thereof that retain the desired activity.

The following sequences correspond to the V1 and V5 domains of δPKC and to exemplary fragments derived therefrom. Some exemplary modified peptides are also described below, where the substitution(s) are indicated in lower case. In all cases, it is appreciated that sequences derived from and homologous to those expressly indicated herein (e.g., closely homologous sequences from other species) are contemplated. All peptides described herein can be prepared by chemical synthesis using either automated or manual solid phase synthetic technologies, known in the art. The peptides can also be prepared recombinantly, using techniques known in the art.

A table of preferred δPKC inhibitory peptides is provided below.

TABLE 1
Exemplary δPKC V1 Inhibitory Peptides

| Peptide | SEQ ID NO | Peptide Sequence |
|---|---|---|
| δV1-1.1 | SEQ ID NO: 1 | S-F-N-S-Y-E-L-G-S-L |
| δV1-1.2 | SEQ ID NO: 2 | T-F-N-S-Y-E-L-G-S-L |
| δV1-1.3 | SEQ ID NO: 3 | A-F-N-S-N-Y-E-L-G-S-L |
| δV1-1.4 | SEQ ID NO: 4 | S-F-N-S-Y-E-L-G-T-L |
| δV1-1.5 | SEQ ID NO: 5 | S-T-N-S-Y-E-L-G-S-L |
| δV1-1.6 | SEQ ID NO: 6 | S-F-N-S-F-E-L-G-S-L |
| δV1-1.7 | SEQ ID NO: 7 | S-N-S-Y-D-L-G-S-L |
| δV1-1.8 | SEQ ID NO: 8 | S-F-N-S-Y-E-L-P-S-L |
| δV1-1.9 | SEQ ID NO: 9 | T-F-N-S-Y-E-L-G-T-L |
| δV1-1.10 | SEQ ID NO: 10 | S-F-N-S-Y-E-I-G-S-V |
| δV1-1.11 | SEQ ID NO: 11 | S-F-N-S-Y-E-V-G-S-I |
| δV1-1.12 | SEQ ID NO: 12 | S-F-N-S-Y-E-L-G-S-V |
| δV1-1.13 | SEQ ID NO: 13 | S-F-N-S-Y-E-L-G-S-I |
| δV1-1.14 | SEQ ID NO: 14 | S-F-N-S-Y-E-I-G-S-L |
| δV1-1.15 | SEQ ID NO: 15 | S-F-N-S-Y-E-V-G-S-L |
| δV1-1.16 | SEQ ID NO: 16 | A-F-N-S-Y-E-L-G-S-L |
| δV1-1.17 | SEQ ID NO: 17 | Y-D-L-G-S-L |
| δV1-1.18 | SEQ ID NO: 18 | F-D-L-G-S-L |
| δV1-1.19 | SEQ ID NO: 19 | Y-D-I-G-S-L |
| δV1-1.20 | SEQ ID NO: 20 | Y-D-V-G-S-L |
| δV1-1.21 | SEQ ID NO: 21 | Y-D-L-P-S-L |
| δV1-1.22 | SEQ ID NO: 22 | Y-D-L-G-L-L |
| δV1-1.23 | SEQ ID NO: 23 | Y-D-L-G-S-I |
| δV1-1.24 | SEQ ID NO: 24 | Y-D-L-G-S-V |
| δV1-1.25 | SEQ ID NO: 25 | I-G-S-L |
| δV1-1.26 | SEQ ID NO: 26 | V-G-S-L |
| δV1-1.27 | SEQ ID NO: 27 | L-P-S-L |
| δV1-1.28 | SEQ ID NO: 28 | L-G-L-L |
| δV1-1.29 | SEQ ID NO: 29 | L-G-S-I |
| δV1-1.30 | SEQ ID NO: 30 | L-G-S-V |
| δV1-1.31 | SEQ ID NO: 31 | E-L-G-S-L-Q-A-E-D-D |
| δV1-1.32 | SEQ ID NO: 32 | E-L-G-S-L-Q-A-E-D-E |
| δV1-1.33 | SEQ ID NO: 33 | S-F-N-S-Y-E-L-G-S |
| δV1-1.34 | SEQ ID NO: 34 | S-F-N-S-Y-E-L-G-S-L |
| δV1-1.35 | SEQ ID NO: 35 | E-L-G-S-L-Q-A-E-D-D |
| δV1-1.36 | SEQ ID NO: 36 | G-S-F-N-S-Y-E-L-G-S-L-G-G |
| δV1-1.37 | SEQ ID NO: 37 | S-A-N-S-Y-E-L-G-S-L |
| δV1-1.38 | SEQ ID NO: 38 | N-S-Y-E-L-G-S-L |
| δV1-2 | SEQ ID NO: 39 | A-L-S-T-E-R-G-K-T-L-V |
| δV1-2.1 | SEQ ID NO: 40 | A-L-S-T-D-R-G-K-T-L-V |
| δV1-2.2 | SEQ ID NO: 41 | A-L-T-S-D-R-G-K-T-L-V |
| δV1-2.3 | SEQ ID NO: 42 | A-L-T-T-D-R-G-K-S-L-V |
| δV1-2.4 | SEQ ID NO: 43 | A-L-T-T-D-R-P-K-T-L-V |
| δV1-2.5 | SEQ ID NO: 44 | A-L-T-T-D-R-G-R-T-L-V |
| δV1-2.6 | SEQ ID NO: 45 | A-L-T-T-D-K-G-K-T-L-V |
| δV1-2.7 | SEQ ID NO: 46 | A-L-T-T-D-K-G-K-T-L-V |
| δV1-3 | SEQ ID NO: 47 | V-L-M-R-A-A-E-E-P-V |
| δV1-4 | SEQ ID NO: 48 | Q-S-M-R-S-E-D-E-A-K |
| δV1-5 | SEQ ID NO: 49 | A-F-N-S-Y-E-L-G-S |
| δV3-1 | SEQ ID NO: 50 | Q-G-F-E-K-K-T-G-V |
| δ3-2 | SEQ ID NO: 51 | D-N-N-G-T-Y-G-K-I |
| δV5-1 | SEQ ID NO: 52 | K-N-L-I-D-S |
| δ5-2 | SEQ ID NO: 53 | V-K-S-P-R-D-Y-S |
| δV5-2.1 | SEQ ID NO: 54 | V-K-S-P-C-R-D-Y-S |
| δ5-2.2 | SEQ ID NO: 55 | I-K-S-P-R-L-Y-S |
| δ5-3 | SEQ ID NO: 56 | K-N-L-I-D-S |
| δ5-4 | SEQ ID NO: 57 | P-K-V-K-S-P-R-D-Y-S-N |
| δV1-1.1 | SEQ ID NO: 58 | S-F-N-S-Y-E-L-G-S-L |
| δV1-1.2 | SEQ ID NO: 59 | T-F-N-S-Y-E-L-G-S-L |
| δV1-1.3 | SEQ ID NO: 60 | A-F-N-S-N-Y-E-L-G-S-L |
| δV1-1.4 | SEQ ID NO: 61 | S-F-N-S-Y-E-L-G-T-L |
| δV1-1.5 | SEQ ID NO: 62 | S-T-N-S-Y-E-L-G-S-L |
| δV1-1.6 | SEQ ID NO: 63 | S-F-N-S-F-E-L-G-S-L |
| δV1-1.7 | SEQ ID NO: 64 | S-N-S-Y-D-L-G-S-L |
| δV1-1.8 | SEQ ID NO: 65 | S-F-N-S-Y-E-L-P-S-L |
| δV1-1.9 | SEQ ID NO: 66 | T-F-N-S-Y-E-L-G-T-L |
| δV1-1.10 | SEQ ID NO: 67 | S-F-N-S-Y-E-I-G-S-V |
| δV1-1.11 | SEQ ID NO: 68 | S-F-N-S-Y-E-V-G-S-I |
| δV1-1.12 | SEQ ID NO: 69 | S-F-N-S-Y-E-L-G-S-V |
| δV1-1.13 | SEQ ID NO: 70 | S-F-N-S-Y-E-L-G-S-I |
| δV1-1.14 | SEQ ID NO: 71 | S-F-N-S-Y-E-I-G-S-L |
| δV1-1.15 | SEQ ID NO: 72 | S-F-N-S-Y-E-V-G-S-L |
| δV1-1.16 | SEQ ID NO: 73 | A-F-N-S-Y-E-L-G-S-L |
| δV1-1.17 | SEQ ID NO: 74 | Y-D-L-G-S-L |
| δV1-1.18 | SEQ ID NO: 75 | F-D-L-G-S-L |
| δV1-1.19 | SEQ ID NO: 76 | Y-D-I-G-S-L |
| δV1-1.20 | SEQ ID NO: 77 | Y-D-V-G-S-L |
| δV1-1.21 | SEQ ID NO: 78 | Y-D-L-P-S-L |
| δV1-1.22 | SEQ ID NO: 79 | Y-D-L-G-L-L |
| δV1-1.23 | SEQ ID NO: 80 | Y-D-L-G-S-I |
| δV1-1.24 | SEQ ID NO: 81 | Y-D-L-G-S-V |
| δV1-1.25 | SEQ ID NO: 82 | I-G-S-L |
| δV1-1.26 | SEQ ID NO: 83 | V-G-S-L |
| δV1-1.27 | SEQ ID NO: 84 | L-P-S-L |
| δV1-1.28 | SEQ ID NO: 85 | L-G-L-L |
| δV1-1.29 | SEQ ID NO: 86 | L-G-S-I |
| δV1-1.30 | SEQ ID NO: 87 | L-G-S-V |
| δV1-2 | SEQ ID NO: 88 | A-L-S-T-E-R-G-K-T-L-V |
| δV1-2.1 | SEQ ID NO: 89 | A-L-S-T-D-R-G-K-T-L-V |
| δV1-2.2 | SEQ ID NO: 90 | A-L-T-S-D-R-G-K-T-L-V |
| δV1-2.3 | SEQ ID NO: 91 | A-L-T-T-D-R-G-K-S-L-V |
| δV1-2.4 | SEQ ID NO: 92 | A-L-T-T-D-R-P-K-T-L-V |
| δV1-2.5 | SEQ ID NO: 93 | A-L-T-T-D-R-G-R-T-L-V |
| δV1-2.6 | SEQ ID NO: 94 | A-L-T-T-D-K-G-K-T-L-V |
| δV1-2.7 | SEQ ID NO: 95 | A-L-T-T-D-K-G-K-T-L-V |
| δV1-3 | SEQ ID NO: 96 | V-L-M-R-A-A-E-E-P-V |
| δV1-4 | SEQ ID NO: 97 | Q-S-M-R-S-E-D-E-A-K |
| δV1-5 | SEQ ID NO: 98 | A-F-N-S-Y-E-L-G-S |
| δV3-1 | SEQ ID NO: 99 | Q-G-F-E-K-K-T-G-V |
| δ3-2 | SEQ ID NO: 100 | D-N-N-G-T-Y-G-K-I |
| δ5-1 | SEQ ID NO: 101 | K-N-L-I-D-S |
| δ5-2 | SEQ ID NO: 102 | V-K-S-P-R-D-Y-S |
| δ5-2.1 | SEQ ID NO: 103 | V-K-S-P-C-R-D-Y-S |
| δ5-2.2 | SEQ ID NO: 104 | I-K-S-P-R-L-Y-S |
| δ5-3 | SEQ ID NO: 105 | K-N-L-I-D-S |
| δ5-4 | SEQ ID NO: 106 | P-K-V-K-S-P-R-D-Y-S-N |

KAI-9803 is a selective δPKC inhibitor that has been shown to be an effective inhibitor of δPKC activity. As such, this peptide construct represents a preferred embodiment. The term “KAI-9803” refers to an peptide derived from the first variable region of δPKC conjugated via a Cys-Cys disulfide linkage to a HIV Tat-derived transporter peptide, and can be represented as follows:

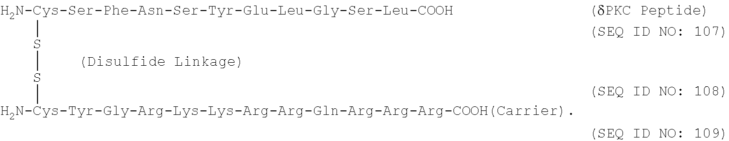

Additional embodiments of inhibitory peptides are provided below:

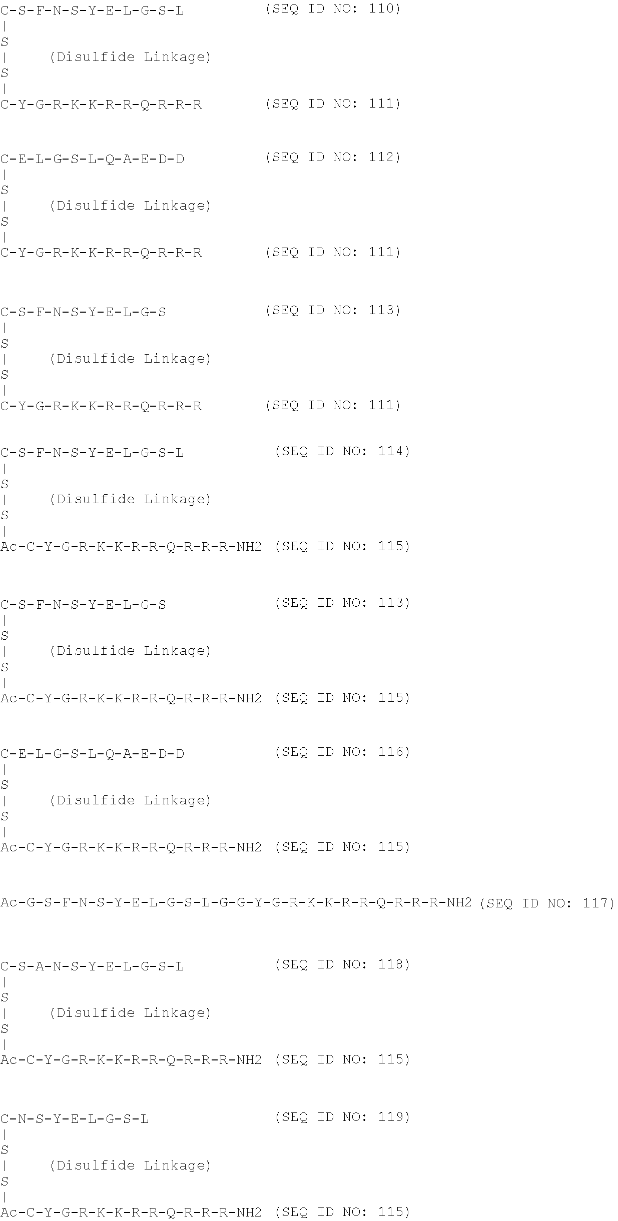

Preclinical models where KAI-9803 has shown a benefit include in vitro global cardiac ischemia and reperfusion in rats (Inagaki K., et al., Circulation 2003 pp: 869), in vivo local left anterior descending (LAD) coronary artery occlusion and reperfusion in pigs (Inagaki K., et al., Circulation 2003 pp:2304) and in vivo middle cerebral artery occlusion (MCAO) in rats (Bright R., et al., J. Neuroscience 2004). Due to the promising efficacy KAI-9803 demonstrated in preclinical models of reperfusion injury, KAI Pharmaceuticals further investigated the effects of KAI-9803 in an animal model of ischemic stroke to determine if inhibition of development of δPKC would also be effective in protecting the brain form ischemia reperfusion induced damage with intravenous bolus or infusion administration. Both transient and permanent middle cerebral occlusion (MCAO) models of ischemic stroke in rats were used to test the dose response potency and efficacy of KAI-9803.

The V5 domain of the .delta.PKC isozyme has the amino acid sequence: “PKVKSPRDY SNFDQEFLNE KARLSYSDKN LIDSMDQSAF AGFSFVNPKF EHLLED” (SEQ ID NO:120). Exemplary peptides include VKSPRDYS (SEQ ID NO:121) taken from amino acid residues 624-631, PKVKSPRDY SN (SEQ ID NO:122), and modified peptides VKSPcRDYS (SEQ ID NO:123) and iKSPR.sub.1YS (SEQ ID NO:124).

### Carrier Peptides

The term “carrier” refers to a moiety that facilitates cellular uptake, such as cationic polymers, peptides and antibody sequences, including polylysine, polyarginine, Antennapedia-derived peptides, HIV Tat-derived peptides and the like, as described, for example, in US Publications Nos. and U.S. Pat. Nos. 4,847,240, 5,888,762, 5,747,641, 6,316,003, 6,593,292, US2003/0104622, US2003/0199677 and US2003/0206900. Another well known carrier peptide sequence is the “poly-Arg” sequence. See, e.g., U.S. Pat. No. 6,306,993, which is also hereby incorporated by reference in its entirety. An example of a carrier moiety is a “carrier peptide,” which is a peptide which facilitates cellular uptake of an δPKC inhibitory peptide which is chemically associated or bonded to the transporter peptide.

In many cases, a disulfide bond is used to link the carrier and cargo peptides, producing the therapeutic peptide construct. In such embodiments, cargo and carrier peptides are linked via Cys disulfide bonds. The Cys residues can be located at the N-terminus, the C-terminus, or internal to the peptides. Another strategy to improve peptide composition stability involves joining the cargo and carrier peptides into a single peptide as opposed to joining the peptides via a disulfide bond. An exemplary carrier peptide is YGRKKRRQRRR (SEQ ID NO:125). Examples of modification to the disulfide bonds and other linking strategies are discussed in U.S. Provisional Application Nos. 60/881,419 and 60/945,285, which are both hereby incorporated by reference in their entirety.

### Administration

The peptides are prepared for administration by combining with a pharmaceutically-acceptable carrier or diluent. Thus, a further aspect of the invention provides pharmaceutical compositions comprising a peptide of the invention in a dosage form for administration to a subject. Such a dosage form includes, but is not limited to, tablets, capsules, suspensions, syrups for oral administration, where suitable pharmaceutical carriers include mannitol, glucose, starch, lactose, talc, magnesium stearate, aqueous solutions, oil-water emulsions, and the like. Other dosage forms include intrathecal, intravenous, intramuscular, subcutaneous, where suitable pharmaceutical carriers include buffered-aqueous or non-aqueous media. Exemplary formulations can be found in U.S. Pat. No. 7,265,092, which is hereby incorporated by reference in its entirety. The peptides can be locally administered (e.g., near a site of inflammation or peripheral nerve damage) for example, by topical application, intradermal injection or drug delivery catheter.

The amount of the peptide in the composition can be varied so that a suitable dose is obtained and a therapeutic effect is achieved. The dosage will depend on a number of factors such as the route of administration, the duration of treatment, the size and physical condition of the patient, the potency of the peptide and the patient's response. Effective amounts of the peptide can be determined by testing the peptide in one or more models known in the art, including those described herein.

The peptides can be administered as needed, hourly, several times per day, daily, or as often as the person experiencing the pain or that person's physician deems appropriate. The peptides can be administered on an on-going basis for management of chronic indications, or can be administered on a short term basis prior to after an acute indications.

The peptides of the invention can be administered alone or linked to a carrier peptide, such as a Tat carrier peptide. Other suitable carrier peptides are known and contemplated, such as the Drosophila Antennapedia homeodomain (Theodore, L., et at. J. Neurosci. 15:7158 (1995); Johnson, J. A., et al., Circ. Res. 79:1086 (1996b)), where the PKC peptide is cross-linked via an N-terminal Cys-Cys bond to the Antennapedia carrier. Polyarginine is another exemplary carrier peptide (Mitchell et al., J. Peptide Res., 56:318-325 (2000); Rothbard et al., Nature Med., 6:1253-1257 (2000)).

### Method of Use

Without being limited to any particular mode of action, the peptides of the invention are thought to act as translocation inhibitors of δPKC to prevent cell damage due to traumatic brain injury.

It will be appreciated that the peptides can be used in native form or modified by conjugation to a carrier, such as those described above. Alternatively, one or two amino acids from the sequences can be substituted or deleted and exemplary modifications and derivatives and fragments for each peptide are given below.

Where the peptide is part of a conjugate, the peptide is typically conjugated to a carrier peptide, such as Tat-derived transport polypeptide, polyarginine, or Antennapedia peptide by a Cys-Cys bond. In another general embodiment, the peptides can be introduced to a cell, tissue or whole organ using a carrier or encapsulant, such as a liposome in liposome-mediated delivery.

The peptide may be (i) chemically synthesized or (ii) recombinantly produced in a host cell using, e.g., an expression vector containing a polynucleotide fragment encoding said peptide, where the polynucleotide fragment is operably linked to a promoter capable of expressing mRNA from the fragment in the host cell.

In another aspect, the invention includes a method of reducing traumatic brain injury, in a preferred embodiment the traumatic brain injury does not include TBI caused by stroke. The method includes introducing a therapeutically-effective amount of an isozyme-specific δPKC antagonist, or any of the modification, derivatives, and fragments of these peptides described above. The δPKC antagonist inhibits δPKC, resulting in protection of the brain cell, tissue or whole organ by reducing TBI. The reduction of TBI is measured relative to the injury suffered by a corresponding brain cells, tissues or the whole organ that did not undergo δPKC antagonist peptide treatment.

It will be appreciated that the dose of peptide administered will vary depending on the condition of the subject, the timing of administration (that is, whether the peptide is administered prior to, during, or after a TBI inducing event). Those of ordinary skill in the art are able to determine appropriate dosages, using, for example, the dosages used in the whole organ and animal studies described herein.

The method can be practiced with a variety of central nervous system (CNS) cells (e.g., neurons, glial cells).

The peptides can be administered to the cell, tissue or whole organ in vitro, in vivo, or ex vivo. All modes of administration are contemplated, including intraveneous, parenteral, subcutaneous, inhalation, intranasal, sublingual, mucosal, and transdermal. A preferred mode of administration is by infusion or reperfusion through arteries to a target organ.

The following examples are offered to illustrate but not to limit the invention.

### EXAMPLE 1

### Impact of Delta PKC Inhibitors on Stroke TBI

Previous studies have shown that a selective KAI-9803 reduces cerebral infarct size in a transient middle cerebral artery occlusion (MCAO) stroke model in rats evaluated at 24 hours of reperfusion. The goal of this study is to evaluate the ability of a short treatment with KAI-9803 at dose of 13.4mg/kg via intravenous (IV) bolus injection to provide prolonged protective effects by inhibiting cellular injury or inflammatory reaction or by promoting astrocyte proliferation in a rat transient MCAO model during 7 days of recovery.

Short treatment with KAI-9803 resulted in 31% reduction (36.7% of treatment group vs. 53.2% of saline group, p<0.01) of infarct size at 24hrs of reperfusion. Further reduction in infarct size was observed at 3 and 7 days of reperfusion in KAI-9803-treated groups but not in saline group (32.9% vs. 50.2% of infarct size at day-3 and 25.3% vs. 50.0% of infarct size at day-7, p<0.01). Treatment of KAI-9803 protected against neuron and astrocyte damage in penumbra observed at 1 day of reperfusion (303±25/mm² vs. 105±12/mm² for neurons, 132±21/mm² vs. 78±8/mm² for astrocytes, p<0.01). Macrophage infiltration in ischemia core and penumbra occurred after 1 day of reperfusion and increased significantly till 7 days of reperfusion. Treatment of KAI-9803 resulted in 50% reduction of macrophage infiltration in penumbra over 7 days. KAI-9803 protected against capillary damage due to ischemia reperfusion injury in ischemia core and penumbra at 1 day of reperfusion and enhanced capillary density reverse over 7 days of reperfusion (capillary density: 269±10/mm² vs. 178±6/mm² in penumbra and 375±26/mm² vs. 248±15/mm² in ischemia core at day-3 of reperfusion). Astrocyte proliferation (Ki67-positive astrocytes) in penumbra was significantly higher in KAI-9803-treated group than in saline control (p<0.01) after 3 days of reperfusion. Data demonstrated that KAI-9803 not only reduces the brain cellular injury, capillary damage and macrophage infiltration in penumbra at 1 day of reperfusion, but also promotes astrocyte proliferation both in ischemia core and penumbra, enhances capillary density recovery in ischemia core and penumbra which may contribute to the healing of necrotic brain tissue, showing a prolonged protective effect of KAI-9803 for ischemic stroke.

### Material and Methods

### 1. Test Compound

KAI-9803 API (Lot No. U0703AI, peptide content 80%) was obtained from American Peptide Company and stored in a medical freezer set at −20° C.

### 2. Animals

Male Sprague Dawley rats were purchased from Charles River Laboratories (requested purchase weight 250-275 g). Animals were maintained in a temperature-controlled environment with a constant natural 12 hours light/12 hours dark cycle and adequate food and water at all times. All experimental procedures with animals were performed according to IACUC guidelines in the AKI animal research facility.

### 3. Operation Procedure

### Animal Anesthesia

General anesthesia was induced by inhalation Isoflurane at 2.5% with Oxygen and maintained at 2.5% Isoflurane with Oxygen throughout operative procedures.

### Middle Cerebral Artery Occlusion

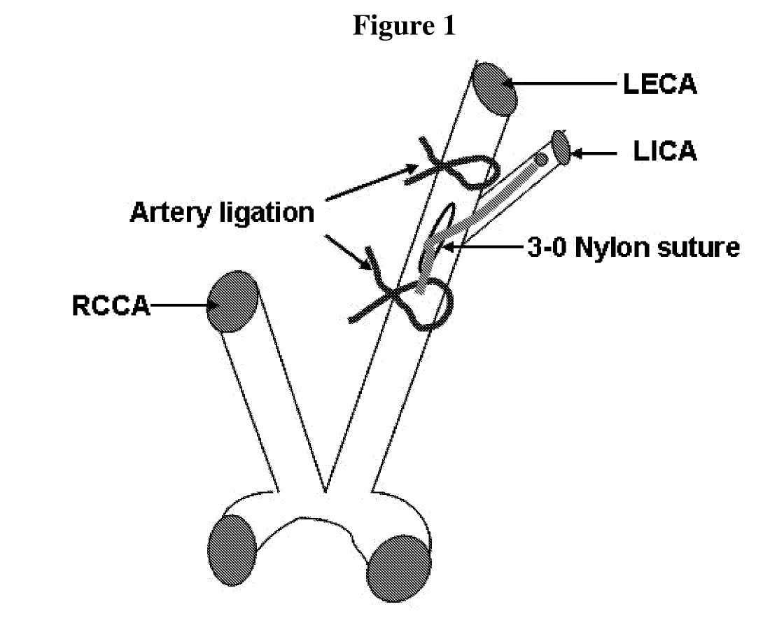

A neck mid ventral skin incision was made and the musculature divided and retracted to expose the left carotid artery. With the aid of a surgical microscope, the external and internal branches of left carotid artery were visualized. The external carotid artery was doubly ligated. Left common carotid artery proximal to the bifurcation was ligated and an arteriotomy was performed distal to the ligation. An occlusive 3-0 monofilament thread was advanced into the left internal carotid branch so that the tip of the monofilament was positioned at the origin of the left middle cerebral artery and occludes arterial blood flow into the left middle cerebral artery (FIG. 1). The occluding thread was sutured in place to occlude the vessel. Suture was permanent or temporary depending on the designed experiment. Retraction was removed, the muscle groups were approximated, and the skin incision was closed with 2-0 silk suture. For temporary ischemia (the reperfusion model), the occlusive 3-0 monofilament was removed after 2 hours of ischemia, reperfusion model was created. Physiological parameters including body temperature (36-38° C.) and respiration rate were monitored and maintained using a heat blanket and/or anesthetic adjustment during the ischemic period.

### Postoperative Management

After the operation, the incision was sutured, and the rat's fur was swabbed with wet gauze to remove blood and carefully dried. The rat was returned to the holding area and placed in a cage by itself for at least 4 hours with water and food. The rat was observed closely until it recovered from surgery, and then was transferred to a cage until up to other rats for the duration of the experiment.

### 4. Drug Administration

Test article was reconstituted in saline and administered by intravenous (IV) bolus injection through the tail vein. The volume of injection was 1.0 mL to limit effects on the volume status of the animal.

### 5. Experiment Groups

Two groups were designed for the current studies. Group 1 was IV bolus administration of saline via tail vein at the onset of reperfusion in transient MCAO model. All animals were random divided into 3 groups; 1, 3, and 7 days of reperfusion. Group 2 was IV bolus administration of KAI-9803 at dose of 13.4mg/kg via tail vein at the onset of reperfusion in transient MCAO model. All animals were random divided into 3 groups; 1, 3, and 7 days of reperfusion.

### 6. Animal Sacrifice and Brain Sample Preparation

At the end of the experiment, animals with 1 day of reperfusion both in saline and KAI-9803 treatment groups were sacrificed by opening the chest under deep anesthesia (5% Isoflurane), thereby inducing respiratory and cardiac arrest. Brains were carefully moved out and sliced into 5 pieces of equal thickness (2.5 mm in thickness; labeled slices 1-5 with slice #1 corresponding to the front of the brain and #5 corresponding to the back of the brain). All slices were stained with 2% 2,3,5-triphenyltetrazolium chloride (TTC) ex vivo for 10 minutes at room temperature, and stored in 4% Paraformaldehyde (PFA) in 0.1mol/L phosphate buffer (pH 7.4) solution at room temperature (20° C.) for photograph image and histology studies.

Animals with 3 and 7 days of reperfusion both in saline and KAI-9803 treatment groups were received an IP injection of BrdU (50 mg/kg) 1 hr before sacrifice. Animals were then sacrificed by over dose of Pentobarbital IP injection (100 mg/kg), performed for perfusion fixation with 4% PFA solution at room temperature (20° C.) for 10 minutes. After fixation, brains were carefully moved out and sliced into 5 pieces of equal thickness (2.5 mm in thickness). All slices were processed for photograph image and histology studies.

### 7. Histochemical and Immunohistochemical Staining

All brain tissues were processed for paraffin blocks and sectioned as 8-10 μm in thickness. Routine H&E and Toluidine blue staining were performed. Immunohistochemical (IHC) staining for astrocyte (GFAP), macrophage (ED1) and capillary (CE31), IHC for cell proliferation (Ki67 and BrdU) were performed. Ki67/GFAP and BrdU/GFAP double staining was performed for evaluation of astrocyte proliferation.

### 8. Data Analysis

### Infarct Size Calculation

Infarct size at 24 hrs of reperfusion: Data analysis of infarct size of ipsilateral hemisphere brain was determined with same methods as we did before (see stroke report).

Infarct size at day 3 and 7 of reperfusion: H&E stained brain sections (5 pieces, 10 μm thickness) were scanned (HP Scanjet; Model 3970; pixels: 1200) and saved as Photoshop images. Data analysis of infarct size of ipsilateral hemisphere brain was determined with same methods as we did before (see stroke report).

### Cell Calculation

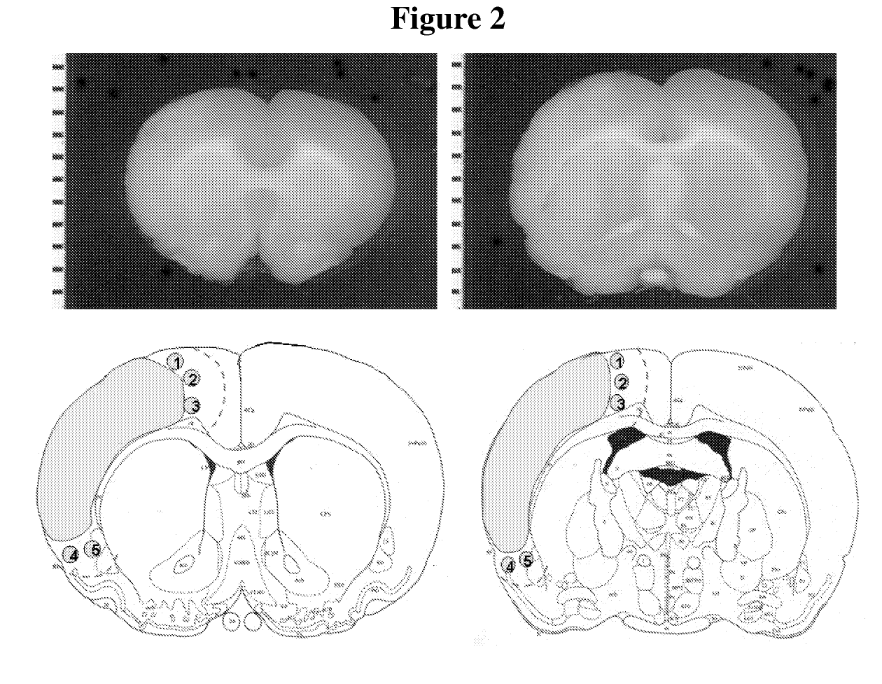

Cell calculation was focused on the cortex area (FIG. 2).

Density of neuron and astrocyte in penumbra: Five different areas of penumbra were selected (high magnification, ×400; 0.08 mm²) from Toluidine blue stained sections for calculation.

### Evaluation of Survival Neuron

The number of normal-appearing neurons was manually counted. Normal neurons were defined as neurons with pale nuclei, whether or not possessing darkened cytoplasm. Neurons showing darkened cytoplasm and nuclei, with or without pyknosis, were interpreted as damaged neurons and were excluded. Survival neuron was defined as the number of neurons per mm² of brain tissue.

### Evaluation of Survival Astrocyte

The number of normal-appearing astrocytes was manually counted. Normal astocytes were defined as oval-appearing nuclei with a narrow rim of heterochromatin and an inconspicuous cytoplasm. Astrocyte was defined as the number of astrocyts per mm² of brain tissue.

### Evaluation of the Density of Capillary in Ischemia Core and Penumbra

Five different areas of ischemia core and penumbra were selected (high magnification, ×400; 0.08 mm²) from CD31 IHC stained sections for calculation. The number of CD31 positive stained capillaries was manually counted. Capillary was defined as the number of CD31-positive capillaries per mm² of brain tissue.

### Evaluation of Macrophage Infiltration in Penumbra

Five different areas of penumbra were selected (high magnification, ×400; 0.08 mm²) from ED1 MC stained sections for calculation. The number of ED1 positive stained cells was manually counted. Macrophage was defined as the number of ED1-positive cells per mm² of brain tissue.

### Astrocyte Proliferation

Evaluation of the astrocyte proliferation in penumbra: Five different areas of penumbra were selected (high magnification, ×400; 0.08 mm²) from Ki67/GFAP and BrdU/GFAP double IHC stained sections for calculation.

The number of Ki-67 positive stained astrocyte was manually counted. Proliferating astrocyte was defined as the number of Ki-67/GFAP-positive cells per mm² of brain tissue.

The number of BrdU positive stained astrocyte was manually counted. In situ proliferating astrocyte was defined as the number of BrdU/GFAP-positive cells per mm² of brain tissue.

### 9. Exclusions

Animals were excluded from the analysis if they died during the surgery or reperfusion period, or if the infarct size was very small (<10%) suggesting that the occlusion (ischemia) was not effective.

### 10. Statistical Analysis

Data from each group was averaged and standard deviations and standard error of the mean (=stdev/sqrt(n)) will be determined. Dosing groups were compared to control group by one-way ANOVA with a post-hoc Dunnett test. P<0.05 was considered statistically significant.

### Results

Effect of KAI-9803 Treatment at Dose of 13.4 mg/kg as IV Bolus Administration in Transient MCAO Model

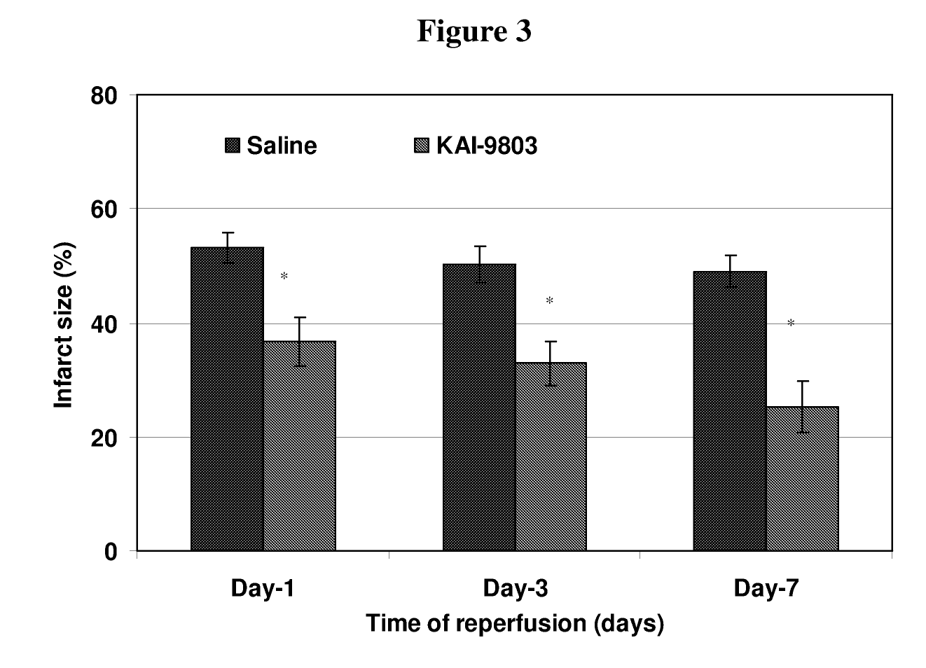

Rats subjected to 2 hrs of left MCAO followed by 1 day of reperfusion were treated with KAI-9803 via tail vein bolus injection at the onset of reperfusion. Rats treated with 13.4 mg/kg of KAI-9803 showed a statistically significant reduction in infarct size compared to saline control (36.7% vs. 53.2%, p<0.01) at 24 hrs of reperfusion. Further reduction in infarct size was also observed at 3 and 7 days after reperfusion in KAI-9803-treated groups but not in saline group (32.9% vs. 50.2% of infarct size at day-3 and 25.3% vs. 50.0% of infarct size at day-7, p<0.01), demonstrating the neuron tissue protective effects of KAI-9803 when administered as an IV bolus administration at the onset of reperfusion (FIG. 3).

### Effect of KAI-9803 on Protection Against Neuron and Astrocyte Damage in Penumbra

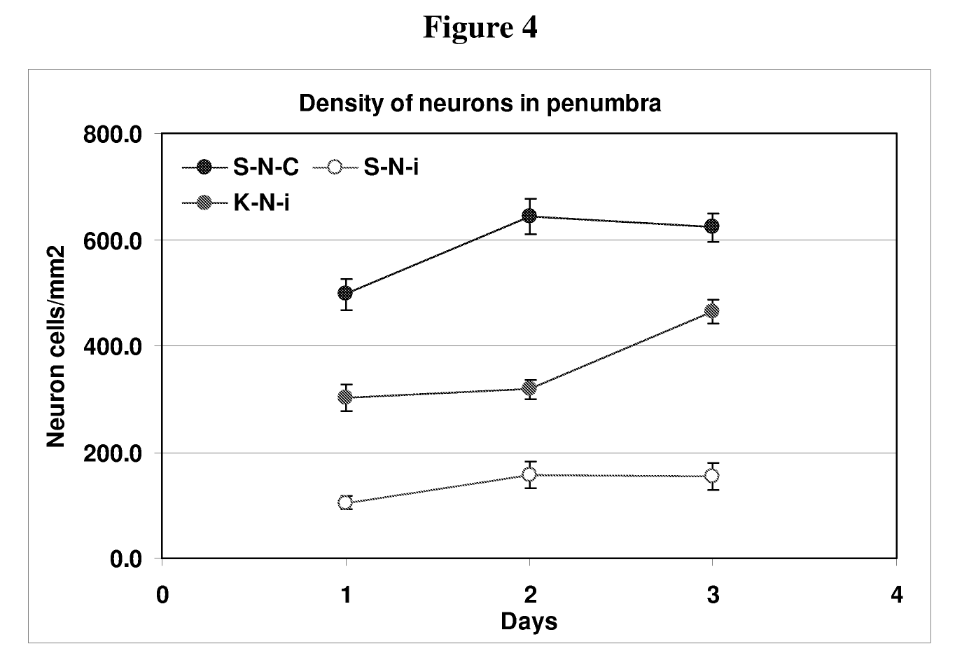

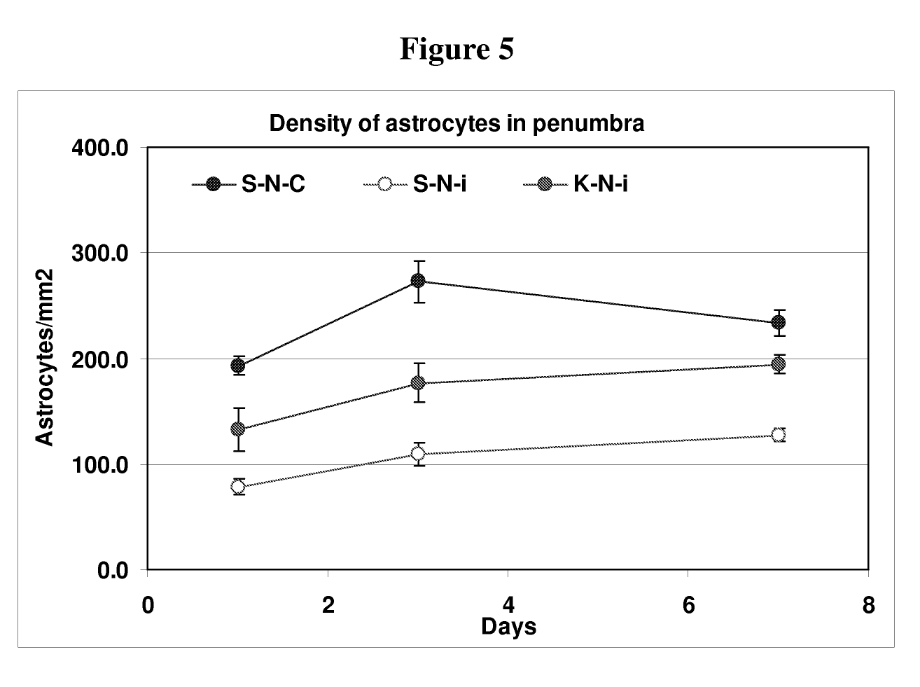

Neuron protection by KAI-9803 treatment was observed in penumbra at 1 day of reperfusion, showing survival neuron density in treated group as 303±25/mm² more than saline treated as105±12/mm² (p<0.01) (FIG. 4). Neuron density in treated group was slightly increased at day 7 of reperfusion, which might be due to the healing of damaged brain tissue. Similarly, astrocyte protection was also observed as 132±21/mm² in treated group than 78±8/mm² in saline group at 1 day of reperfusion (p<0.01) (FIG. 5). After 3 days of reperfusion the density of astrocyte in penumbra was increased significantly in both treated and saline control group, showing much more significantly increase in treated group than in saline group (177±18/mm² vs. 110±11/mm², p<0.01 at day 3; 194±9/mm² vs. 127±6/mm², p<0.01 at day 7).

### Effect of KAI-9803 on Protection Against Capillary Damage in Ischemia Core and Penumbra

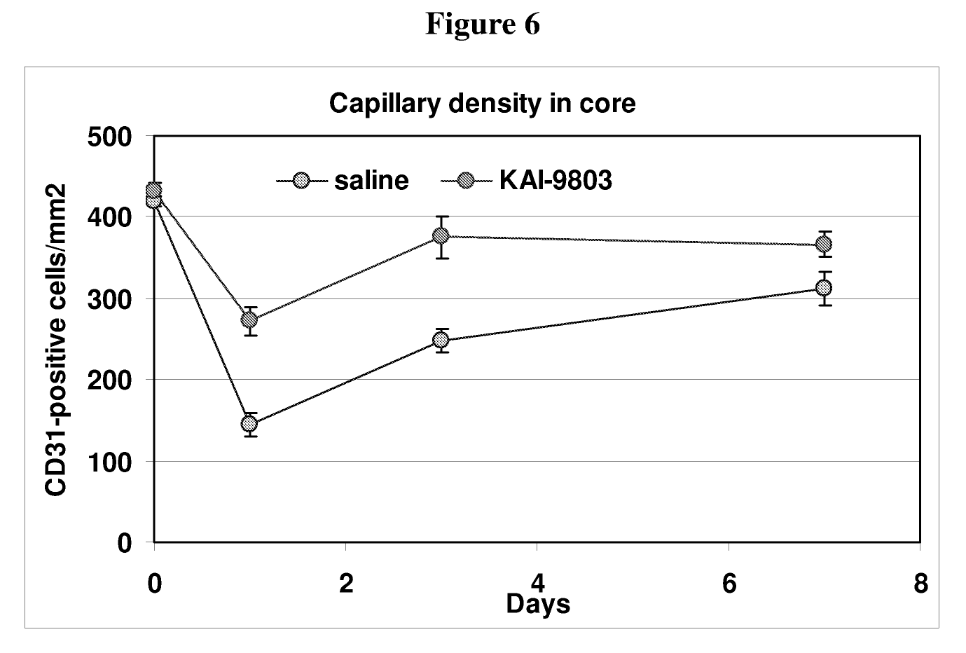

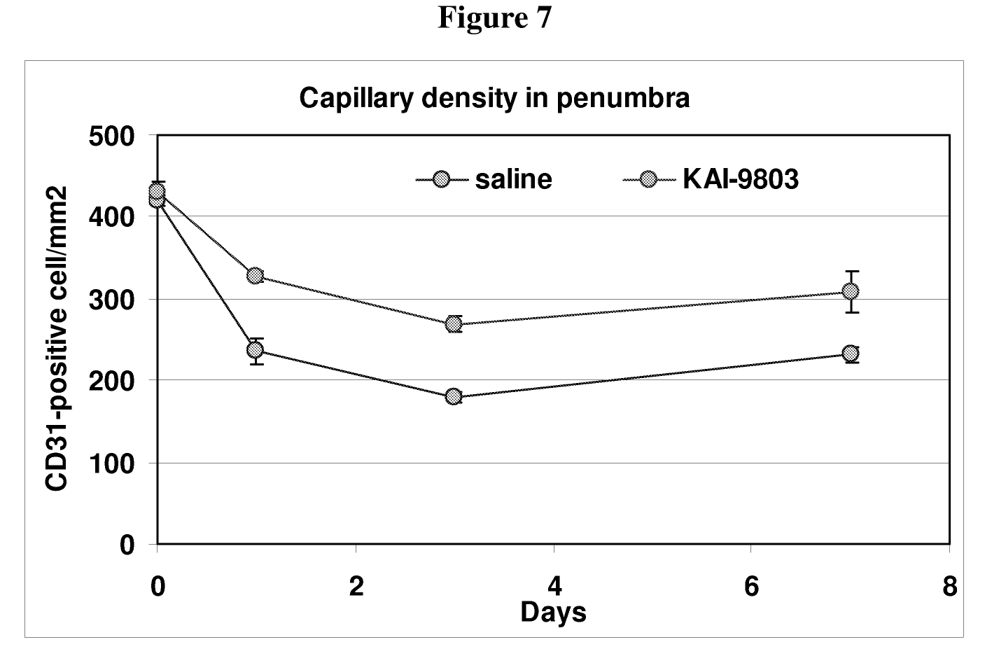

Ischemia-reperfusion induced capillary damage in ischemia core and penumbra was significantly limited by the treatment of KAI-9803. Ischemia-reperfusion injury resulted in a significantly capillary damage in score, showing 419±7/mm² at pre-ischemia vs. 145±15/mm² at 24 hrs of reperfusion in saline group. Treatment of KAI-9803 reduced capillary damage in ischemia core (431±11/mm² at pre-ischemia vs. 272±17/mm² at 24 hrs of reperfusion). Capillary density in ischemia core reversed at 3 and 7 days of reperfusion near pre-ischemia level with the treatment of KAI-9803. However, this reverse was slow and less in saline group (FIG. 6). Capillary density in penumbra showed similar changes as in ischemia core, but less reversing (FIG. 7). These data suggested higher capillary density in ischemia core might be due to the higher angiogenesis in that area. Capillary protection at early time of damage and later angiogenesis would contribute the later damage healing, especially in ischemia core healing.

### Effect of KAI-9803 on Protection Against Macrophage Infiltration in Penumbra

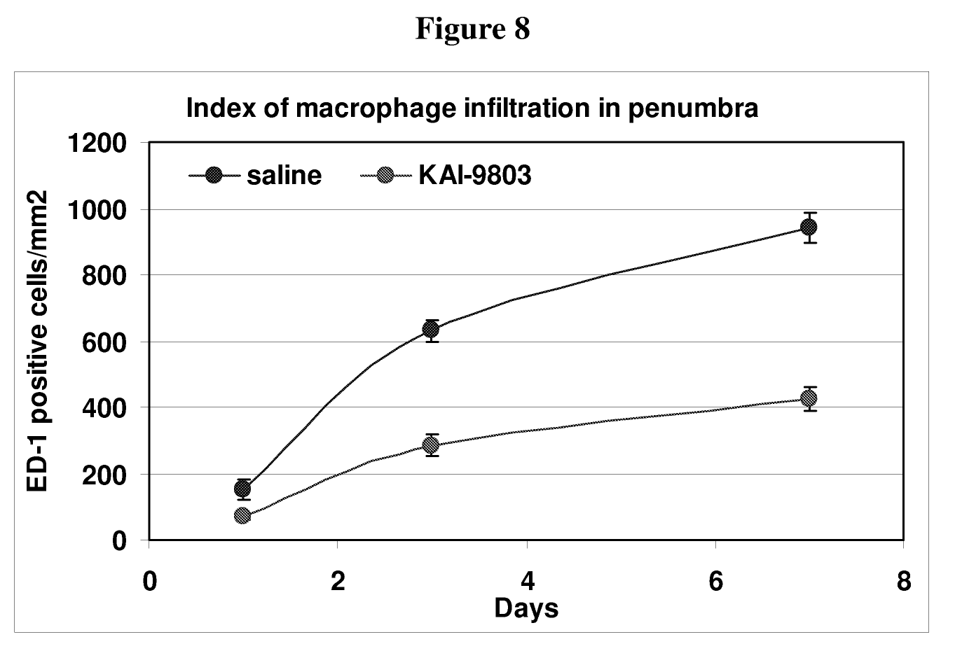

Macrophage infiltration in ischemia core and penumbra was recognized at 1 day of reperfusion. After 3 days of reperfusion, significant macrophage infiltration appeared at the board of ischemia core and penumbra. They were migrating into ischemia core during recovery time. Macrophage infiltration widely appeared in penumbra in saline group as compared with KAI-9803 treated group. The treatment of KAI-9803 resulted in a 50% reduction in macrophage infiltration over 7 days of reperfusion (FIG. 8).

### Treatment of KAI-9803 Enhancement Astrocyte Proliferation in Penumbra

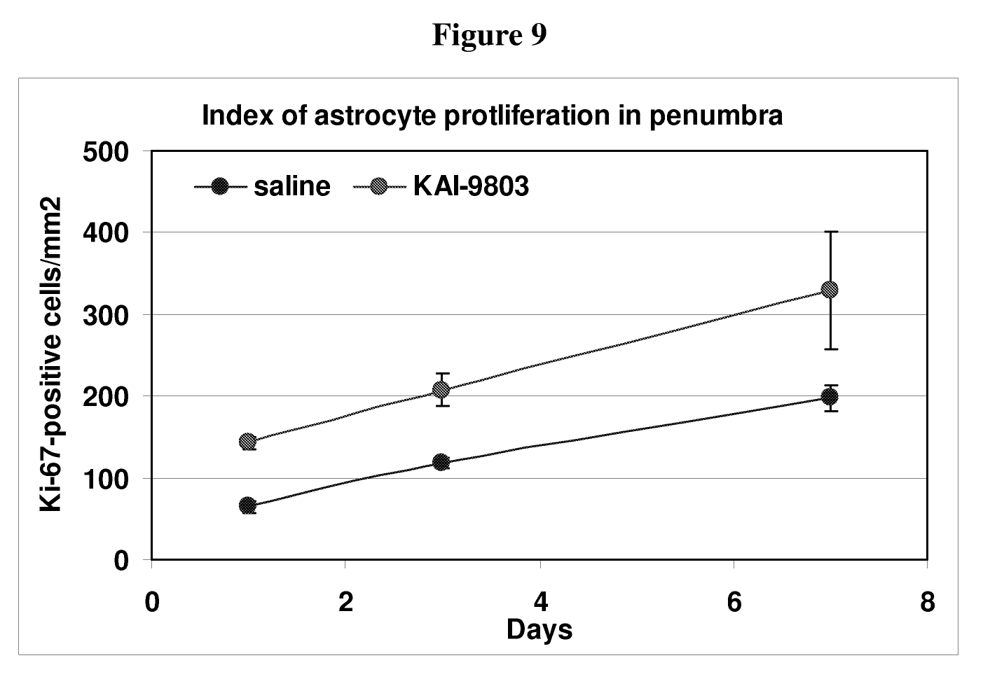

Ki67-positive cells in penumbra indicated a cell proliferation after ischemia reperfusion injury, which cells were mostly astrocytes and other glia cells. Penumbra tissue with KAI-9803 treatment showed more Ki67-positive cells as compared with saline group over 7 days of reperfusion (FIG. 9). In order to understand the index of astrocyte proliferation, double IHC staining of Ki67/GFAP and BrdU/GFAP were performed.

### REFERENCES

1) Inagaki K., et al. Circulation 108:869-875 (2003)

2) Inagaki K., et al. Circulation 108:2304-2307 (2003)

3) Bright R., et al. J. Neuroscience 24(31):6880-6888 (2004)

1. A method to treat traumatic brain injury, comprising:
identifying a subject suffering from a traumatic brain injury (TBI) by identifying the presence of TBI symptoms, and
administering a therapeutically effective amount of a peptide that specifically inhibits δPKC activity, whereby astrocyte activity is increased and one or more of the symptoms of TBI are reduced.

2. The method of claim 1, wherein said administering occurs within 1 to 5 hours of the TBI.

3. The method of claim 1, wherein the peptide comprises 4 to 25 residues of the first variable region of δPKC.

4. The method of claim 3, wherein the peptide comprises 6 to 25 residues of the first variable region of δPKC.

5. The method of claim 1, wherein the peptide comprises 6 to 25 residues of the fifth variable region of δPKC.

6. The method of claim 1, wherein said administering comprises administering the δPKC peptide antagonist linked to a moiety effective to facilitate transport across a cell membrane.

7. The method of claim 6, wherein the moiety is selected from the group consisting of a Tat-derived peptide, an Antennapedia carrier peptide, and a polyarginine peptide.

8. The method of claim 6, wherein the peptide is KAI-9803.

9. The method of claim 1, wherein the symptoms comprise increase of glucose utilization, energy-dependent membrane depolarization, or cerebral metabolic rate changes.

10. A method of stimulating astrocyte activity, comprising:
providing a therapeutically effective amount of a δPKC inhibitory peptide to a population of astrocytes, whereby astrocyte proliferation is increased relative to a population of astrocytes not provided the δPKC inhibitory peptide.

11. The method of claim 10, wherein the peptide comprises 4 to 25 residues of the first variable region of δPKC.

12. The method of claim 10, wherein the peptide is KAI-9803.

13. The method of claim 10, wherein the peptide comprises 4 to 25 residues of the fifth variable region of δPKC.

14. The method of claim 10, wherein said administering comprises administering the δPKC peptide antagonist linked to a moiety effective to facilitate transport across a cell membrane.

15. The method of claim 14, wherein the moiety is selected from the group consisting of a Tat-derived peptide, an Antennapedia carrier peptide, and a polyarginine peptide.
